(19) United States
(12) Patent Application Publication
(10) Pub. No.: US 2004/0192581 A1
(43) Pub. Date: Sep. 30, 2004

(54) Topical anti-infective formulations

(75) Inventors: Scott M. Walsh, Germantown, MD (US); Anjali G. Shah, North Potomac, MD (US); James J. Mond, Silver Spring, MD (US)

Correspondence Address: SYNNESTVEDT & LECHNER, LLP, 2600 ARAMARK TOWER, 1101 MARKET STREET, PHILADELPHIA, PA, 191072950

(73) Assignee: Biosynexus Incorporated, Gaithersburg, MD

(21) Appl. No.: 10/733,046

(22) Filed: Dec. 10, 2003

Related U.S. Application Data
(60) Provisional application No. 60/432,182, filed on Dec. 10, 2002.

(51) Int. Cl. A61K 38/16

(52) U.S. Cl. 514/2

(57) ABSTRACT

Topical compositions containing an effective amount of lysostaphin and/or one or more lantibiotics in a pharmaceutically acceptable carrier for topical application. Methods for treating skin or wound infections, including, but not limited to infected abrasions, skin or surface cuts, burns or surgical incisions or decubiti, with the topical compositions are also disclosed.

### CROSS REFERENCE TO RELATED APPLICATION

This application is based on and claims priority benefit under 35 U.S.C. §119(e) of U.S. Provisional Application Serial No. 60/432,182 filed Dec. 10, 2002. The entire disclosure of this provisional application is relied upon and incorporated by reference herein.

### BACKGROUND OF THE INVENTION

Bacterial skin and wound infections are a significant problem, particularly in institutional settings such as hospitals, nursing homes, schools (especially athletic locker rooms) and infirmaries. Individuals particularly at risk include infants, the elderly, those undergoing in-patient or out-patient invasive procedures (essentially any patient prior to release from a hospital), those suffering from various conditions that predispose them to skin infections, including the presence of foreign bodies, the immuno-compromised, the immuno-suppressed, those convalescing, and those with chronic conditions requiring frequent hospital stays. Among non-human patients, those at risk include zoo animals, herd animals, and animals maintained in close quarters (such as kenneled and stabled animals), and include, but are not limited to, pigs, cattle, sheep, goats, and the like. These individuals are at elevated risk for the skin or wound infection to spread to or otherwise damage other organ systems. Further, the advent of multiple drug resistant strains of pathogens increases the concern and need for timely blocking and treatment of such infections. In the United States alone some 14,000 people are infected and die each year as a result of infection from drug-resistant microbes acquired in hospitals, with the infection often starting as a skin or wound infection. Around the world, as many as 60% of hospital-acquired infections are caused by drug-resistant microbes.

The source of infection is often an individual's own skin flora, or the skin flora of other individuals at the institution. Thus, the incidence of infection could be significantly reduced if an adequate topical prophylactic treatment was available to decolonize the skin flora of those entering an institution experiencing cases of infection. It would be desirable for decolonizing formulations also to have utility in treating wound and skin infections once they have become established.

Staph. aureus and Pseudomonas aeruginosa are among the most frequently encountered pathogens in primary and secondary skin infections. Topical antibiotics such as mupirocin cream and neomycin are not always effective at clearing these infections and the rising prevalence of multi-drug-resistant bacteria has even further reduced the usefulness of traditional antibiotics.

S. aureus is a highly virulent human pathogen. It is the cause of a variety of human diseases, ranging from localized skin infections to life-threatening bacteremia and infections of vital organs. If not rapidly controlled, S. aureus can spread quickly from the initial site of infection to other organs. Although the foci of infection may not be obvious, organs particularly susceptible to infection include the heart valves, kidneys, lungs, bones, meninges and the skin in burn patients.

Pseudomonas aeruginosa is one of the most commonly encountered pathogens in primary and secondary skin infections. Infections are usually acquired from contaminated water and solutions. Types of infections include “swimmer's ear” (contaminated pool water), eye (contact lens solutions), osteomyelitis in children (nail puncture through sneakers), and nosocomial respiratory tract, urinary and wound (especially burn) infections.

Lysostaphin is a potent antimicrobial agent first identified in Staphylococcus simulans (formerly known as S. staphylolyticus). Lysostaphin is a bacterial endopeptidase capable of cleaving the specific cross-linking polyglycine bridges in the cell walls of staphylococci, and is therefore highly lethal thereto. Expressed in a single polypeptide chain, lysostaphin has a molecular weight of approximately 27 kDa.

The cell wall bridges of Staphylococcus aureus, a coagulase positive staphylococcus, contain a high proportion of glycine, therefore lysostaphin is particularly effective in lysing S. aureus. Lysostaphin has also demonstrated the ability to lyse Staphylococcus epidermidis and other staphylococcal strains.

U.S. Pat. No. 6,028,051 to Climo, et al., discloses a method for the treatment of staphylococcal disease. Relatively high doses of lysostaphin of at least 50 and preferably 100 milli-grams of lysostaphin per kilogram of body weight are used for treatment. The relatively high doses of lysostaphin can be used in single dose treatments or multiple-dose treatments. The lysostaphin can be used singularly or in combination with additional antibiotic agents. The '051 patent also discloses that the cloning and sequencing of the lysostaphin gene permits the isolation of variant forms of lysostaphin that have properties similar to or different from those of wild-type lysostaphin.

U.S. Pat. No. 6,315,996 to O'Callaghan, discloses a method for using lysostaphin as an effective antibiotic for topical treatment of staphylococcus corneal infections. U.S. Pat. No. 5,760,026 to Blackburn, et al., discloses a method for using lysostaphin on a wipe to eliminate and cure staphylococcal infections including the cure of mastitis by intramammary infusion. The method is directed to use in dairy cows.

Lantibiotics are pore-forming lanthionines that, in combination with chelating agents or nonionic surfactants, show potent bactericidal activity against both gram-positive and gram-negative organisms. Lantibiotics are a group of antimicrobial peptides produced by bacteria and characterized by post-translational modifications resulting in the dehydration of serine and threonine residues and the formation of intramolecular thioether bridge structures (lanthionine and β-methyl lanthionine) forming lanthionine rings in the peptide chain. The natural utility of the lantibiotics as food preservatives has been harnessed by man in fermented products such as yogurt, cheese, sauerkraut, fermented fish, and the like, for centuries. A 2.5% by weight preparation of the lantibiotic Nisaplin™ has been marketed worldwide as an industrial food preservative for over 50 years.

Nisin is the best characterized and most widely used of the lantibiotics. It is a 32 amino acid, 3.4 kDa cationic peptide with 5 lanthionine rings produced by certain strains of Lactococcus lactis, formerly known as Streptococcus lactis. The microbicidal activity of nisin is associated with the rings resulting from the thioether bridges and with the dehydro residue. Nisin is bactericidal for a broad range of gram-positive bacteria, which can be attributed to at least four modes of action that are believed to combine to:

(i) form pores in the membrane that allow electrolytes and small metabolites to pass, disrupting the cell's osmotic regulation and energy metabolism;

(ii) prevent effective peptidogylcan assembly for cell wall construction, so that the bacterial cell is unable to assemble fresh cell wall structures in order to divide and multiply; and

(iii) interact with the spore coat of spore-forming bacteria to prevent completion of the germination process.

Each of the foregoing independently results in cell death.

While nisin alone has bactericidal activity limited to gram-positive organisms, nisin in combination with chelating agents such as EDTA or nonionic surfactants such as Tween 20 shows potent bactericidal activity against gram-negative organisms, typified by E. coli. Nisin is rapidly bactericidal, with minimum bactericidal concentration (MBC) values usually equal to or twice the minimum inhibitory concentration (MIC) for a given target organism.

U.S. Pat. Nos. 5,691,301 and 5,753,614 disclose nisin and other lantibiotics as having a broad range of bactericidal activity against gram-negative bacteria when combined with a chelating agent. U.S. Pat. No. 5,762,948 discloses the use of nisin and other lantibiotics for the prevention of mastitis in dairy cattle. U.S. Pat. No. 5,804,549 discloses the use of nisin to treat Helicobacter gastro-intestinal infections. U.S. Pat. Nos. 5,866,539 and 5,910,479 disclose formulations of nisin and other lantibiotics for parenteral and sub-cutaneous administration having bactericidal activity against Staphylococcus, Streptococcus and Enterococcus bacteria. U.S. Pat. No. 5,985,823 discloses the use of nisin and other lantibiotics to treat C. difficile infections of the colon.

Decolonizing the skin to prevent infection and treating existing cases of skin and wound infection remains particularly challenging because of the delicate balance between populations of skin flora. The pathogens of skin flora can re-emerge disproportionately in drug-resistant form if this balance is disrupted, for example, by treatment with formulations that lack effectiveness against pathogens, especially if such formulations are more effective against non-pathogens. Many small-molecule antibiotics that have in the past shown promise systemically or during in vitro testing against pathogens have proven inadequate for decolonization or the treatment of skin or wound infections when formulated for topical application to the skin. There remains a need for potent topical formulations capable of decolonizing the skin to prevent pathogen infection and capable of treating existing cases of skin and wound infection.

### SUMMARY OF THE INVENTION

This need is met by the present invention. It has now been discovered that topical formulations can be prepared from lysostaphin and lantibiotics such as nisin that are effective in treating dermatological infections and wound infections without disrupting the balance of non-pathogenic and pathogenic populations of skin flora to an extent that would result in the emergence of drug-resistant pathogen strains. Infected wounds suitable for treatment include, but are not limited to infected skin abrasions, skin or surface cuts, burns and surgical wounds, as well as decubiti.

Therefore, according to one aspect of the present invention, a topical anti-infective composition is provided containing an effective amount of lysostaphin or a lantibiotic in a pharmaceutically acceptable carrier for topical application. According to one embodiment, the amount of lysostaphin or lantibiotic is effective to treat skin or wound infections.

The topical composition can be in the form of a spray, mist, aerosol, lotion, cream, aqueous or non-aqueous solution or liquid, oil, gel, powder, ointment, paste, unguent, emulsion, suspension, and the like, that may optionally be coated on the surface of a topical applicator, such as a bandage, swab, moist woven or non-woven wipe, and the like. Topical cream formulations according to the present invention have been discovered that have good skin compatibility, promote wound healing, independently possess bactericidal activity and enhance the activity of anti-infective active agents. However, the present invention is not limited to creams for topical application. Essentially any pharmaceutical carrier formulation suitable for topical application is within the scope of the present invention.

Lysostaphin and lantibiotics such as nisin have been discovered to produce a synergistic effect when used in combination. That is, the Fractional Inhibitory Concentration of nisin and lysostaphin against staphylococcus has been found to be less than the MIC against staphylococcus that would be expected to be contributed by each antibiotic in a combined formulation based on the known MIC's of both antibiotics alone against staphylococcus. Therefore, the topical anti-infective compositions of the present invention include embodiments that contain both lysostaphin and one or more lantibiotics such as nisin.

Because the topical compositions of the present invention containing lysostaphin or nisin, alone or in combination, are effective in decolonizing populations of skin pathogens and in treating skin and wound infections, another aspect of the present invention provides methods of use for the inventive compositions. According to one embodiment, a method is provided for treating a skin or wound infection by topically applying to a patient in need thereof at the site of infection an effective amount of a topical composition according to the present invention having an amount of lysostaphin or one or more lantibiotics effective to treat skin or wound infections. According to another embodiment, a method of decolonizing skin pathogen populations is provided, by topically applying to a patient in need thereof at a site requiring decolonization an effective amount of a topical composition according to the present invention having an amount of lysostaphin or a lantibiotic effective to decolonize pathogen populations on the surface of the skin.

Methods according to either embodiment include methods using lysostaphin and one or more lantibiotics in combination, including methods using combinations of lysostaphin and nisin. The uses of the topical anti-infective compositions of the present invention exhibit the remarkable ability to decolonize skin pathogen populations to the point of eradication and block subsequent re-colonization for extended periods of time heretofore unknown. The topical anti-infective compositions of the present invention thus serve to inhibit the spread of hospital-acquired bacterial strains within a hospital, as well as among the community at large.

While the present invention is not limited to topical cream or lotion formulations, another aspect of the present invention provides topical cream or lotion formulations that have good skin compatibility, promote wound healing, independently possess bactericidal activity and enhance the activity of anti-infective active agents. According to this aspect of the present invention an anti-infective topical cream or lotion formulation is provided that is an oil-in-water emulsion of an aqueous phase containing ethoxylated partial glycerides of fatty acids, an oil phase containing a hard fat, an anti-infective active ingredient, and an emulsifier that is an inverse emulsion of a water-soluble polymer in an oil phase. The emulsion formulation is well suited for use with lysostaphin and lantibiotics because it emulsifies under conditions that do not denature these proteins.

Regardless of the form of topical delivery, the spread of skin infections is reduced by the inventive compositions and treatment methods, thereby reducing the overall frequency of infection in a general population. The global reduction of infection in a community is important given the emergence of multi-drug-resistant bacterial strains. Reducing the number of new infections in turn reduces the rate at which new resistant strains appear. A more complete appreciation of the invention and many other intended advantages can be obtained by reference to the detailed description of preferred embodiments and claims, which disclose the principles of the invention and the best modes presently contemplated for carrying them out.

### BRIEF DESCRIPTION OF THE DRAWINGS

FIG. 1A shows the efficacy in a skin infection model of lysostaphin against S. aureus in a topical cream formulation according to the present invention;

FIG. 1B shows the efficacy in a skin infection model of nisin against S. aureus and P. aeruginosa in a topical cream formulation according to the present invention;

FIG. 2 shows the dose-response properties for lysostaphin creams according to the present invention in an S. aureus skin infection model, and also the independent bactericidal activity of the cream without lysostaphin;

FIG. 3 compares a lysostaphin topical cream according to the present invention to BACTROBAN Cream in an S. aureus skin infection model;

FIG. 4 depicts in a skin infection model the dose-response properties of a nisin topical cream according to the present invention and the synergistic result that occurs when nisin and lysostaphin are used in combination in a topical cream according to the present invention;

FIG. 5 depicts the efficacy in a skin infection model of a nisin topical cream according to the present invention against P. aeruginosa, and the enhancement of this efficacy when EDTA is added to the formulation;

FIG. 6 depicts the efficacy of a topical cream base according to the present invention formulated without an anti-infective agent against S. aureus in a skin colonization model, and the enhancement of this efficacy resulting from adding DMSO to the formulation;

FIG. 7 depicts the enhancement of the efficacy in a skin colonization model of a topical cream formulation according to the present invention formulated without an agent when other absorption enhancers are added to the cream; and

FIG. 8 depicts the dose-response properties for nisin topical creams according to the present invention against S. aureus in a skin colonization model.

### DETAILED DESCRIPTION OF PREFERRED EMBODIMENTS

The present invention provides topical antimicrobial compositions containing lysostaphin or a lantibiotic such as nisin. The inventive compositions are useful in the prevention and treatment of wound and skin infections caused by any bacteria susceptible to attack by lysostaphin or lantibiotic activity. The skin infection may be primary or secondary. Prevention is attained by topical application of the compositions of the present invention to decolonize skin pathogen populations.

The term “lysostaphin,” as used herein, encompasses any enzyme or anti-staphylococcal agent having proteolytic activity, in vitro and in vivo, against pentaglycine-containing bridges in the cell wall peptidogylcan of staphylococci. Lysostaphin within the scope of the invention encompass: wild-type lysostaphin and related proteins or anti-staphylococcal agents, lysostaphin mutants, variants, fully synthetic and partially synthetic lysostaphins, and recombinantly expressed lysostaphin proteins. Lysostaphin variants may be generated by post-translational processing of the protein (either by enzymes present in a producer's strain or by means of enzymes or reagents introduced at any stage of the process, or by mutation of the structural gene. Mutations may include site-deletion, insertion, point mutations, domain removal and replacement mutations. Lysostaphin includes, for example, Lysostaphin purified from S. simulans, Ambicin L (recombinant lysostaphin produced in Bacillus sphaericus and available from Nutrition 21 (formerly AMBI) of Purchase, N.Y.), and mature Lysostaphin purified from a Lactococcus lactis expression system or an E. coli expression system, and truncated lysostaphin as set forth in co-pending and commonly owned WO 03/82124, specifically incorporated by reference herein.

Recombinant lysostaphins have a greater specific activity, i.e. amount of activity per volume of formulation. Lysostaphin is naturally produced by a bacterium as a pro-enzyme that is later prototypically processed to produce the mature protein. When lysostaphin is isolated from bacteria, both the active form and the less active pro-enzyme form are present in the resulting preparation. The pro-enzyme form is approximately four-fold less active than the mature, active form. Active forms of naturally produced lysostaphin in turn include a heterologous mix of polypeptides. This heterology is attributable to the proteolytic processing of the pro-enzyme of lysostaphin. This proteolytic processing occurs at a number of different sites near the N-terminus of full-length lysostaphin and leads to a heterologous mix of final active lysostaphin molecules. This variability can differ among lysostaphin preparations derived from natural sources.

The presence of less active forms of lysostaphin dilutes out the concentration of active lysostaphin in the preparation, which is further diluted by the presence of the pro-enzyme, thus decreasing the specific activity of a formulation containing naturally derived lysostaphin. In contrast, recombinant lysostaphin preparations contain a single fully active form of lysostaphin. In such a preparation, there is no less active form or pro-enzyme to dilute out the activity of the mature form of lysostaphin. Thus, topical anti-infective compositions that contain recombinant lysostaphin have a higher specific activity than their naturally derived counterparts.

Lantibiotics belong to the class of peptide bacteriocins containing lanthionine rings. Also included among that class, in addition to nisin, are subtilin, epidermin, gallidermin, cinnamycin, duramycin, ancovenin and Pep 5. These lantibiotics are each produced by different microorganisms. However, subtilin obtained from certain cultures of Bacillus subtilis, and epidermin obtained from certain cultures of Staphylococcus epidermidis, have been found to have molecular structures very similar to that of nisin. Because of the molecular similarities, the other lantibiotics will be equally as effective as nisin as anti-infective agents. Thus, the term “lantibiotic” as used herein, encompasses any lanthionine-containing bacteriocin having the ability to inhibit peptidoglycan assembly necessary for bacterial cell wall construction or to form disruptive pores in bacterial cell membranes regardless of the natural bacterial source or whether it is produced recombinantly.

Lantibiotics within the scope of the invention encompass wild-type lantibiotics and the mutants and variants thereof, fully synthetic and partially synthetic lantibiotics, and recombinantly expressed lantibiotics. As with lysostaphin, variants may be generated by post-translational protein processing or by structural gene mutation. Mutations include site-deletion, insertion, point mutations, domain removal and replacement mutations. Nisin, for example, includes native nisin purified from L. lactis, Ambicin N (a purified form of nisin available from Nutrition 21), and mature or variant recombinant nisins such as the over 20 variants generated by replacing lysines with less polar residues available from NIZO Food Research (Netherlands), including nisin variants H27K and H31K.

The bactericidal activity of nisin is ordinarily limited to gram-positive organisms. However, when nisin is formulated with a chelating agent it also shows potent bactericidal activity against gram-negative bacteria. Nisin topical formulations according to the present invention therefore also include embodiments containing from about a 1 to about a 5 mM, up to about a 10 mM concentration of a chelating agent such as EDTA.

The activity of nisin can also be enhanced by formulation with a non-ionic surfactant at concentrations as low as 0.01 wt %. Examples of non-ionic surfactants suitable for use with the present invention include glycerol monolaurate, sucrose esters such as sucrose palmitate, polysorbate 20, TRITON X100, Isoceteth-20, ARLASOLVE 200L, Lauramine oxide, Decylpolyglucose, Phospholipid PTC, MEROXAPOL 105, and the like. Nisin formulations according to the present invention therefore may optionally contain between about 0.01 and about 5.00 wt % of a non-ionic surfactant.

The activity of nisin is also enhanced by carvacrol (2-p-cymenol), an essential oil extracted from oregano and thyme and also produced synthetically. Carvacrol is approved as a flavoring substance by the FDA. Nisin formulations according to the present invention therefore may also optionally contain a concentration of carvacrol between about 0.025 and about 3.0 mM.

Dosage forms for topical administration of the compositions of the invention include various mixtures and combinations that can be applied topically and will permit even spreading and absorption into cutaneous and mucosal surfaces. Examples include sprays, mists, aerosols, lotions, creams, aqueous and non-aqueous solutions or liquids, oils, gels, powders, ointments, pastes, unguents, emulsions and suspensions. The lysostaphin or lantibiotic can be mixed under sterile conditions with a cosmeceutically or pharmaceutically acceptable carrier, and with any excipients, buffers, or propellants that may be required. Topical formulations are prepared by combining lysostaphin or a lantibiotic with conventional pharmaceutical or cosmeceutical diluents or carriers commonly used in topical dry, liquid, cream and aerosol formulations. Both liquids and powders can be delivered as sprays, mists or aerosols. The present invention includes both patient-specific dosages forms, as well as non-patient-specific multi-dosage forms that can be used to decontaminate populations exposed to pathogens such as anthrax (against which nisin has demonstrated activity) as a consequence of a bioterrorism attack.

Powders can be formed with the aid of any suitable powder base, e.g., talc, lactose, starch, and the like. Solutions can be formulated with an aqueous or non-aqueous base, and can also include one or more dispersing agents, suspending agents, solubilizing agents, and the like. Topical gels are prepared using polymers having a molecular weight and level of concentration effective to form a viscous solution or colloidal gel of an aqueous or non-aqueous solution or suspension of lysostaphin or a lantibiotic. Polymers from which topical gels may be prepared include polyphosphoesters, polyethylene glycols, high molecular weight poly(lactic) acids, hydroxypropyl celluloses, chitosan, polystyrene sulfonates, and the like. Chitosan has been found to increase the solubility of nisin and would thus be expected to increase the solubility of other lantibiotics.

Ointments, creams and lotions are formulated, for example, with an aqueous or oily base and addition of a suitable thickening agent, gelling agent, stabilizing agent, emulsifying agent, dispersing agent, suspending agent, or consistency regulating agent, and the like. Bases include water, an alcohol or an oil, such as liquid paraffin, mineral oil, or a vegetable oil, such as peanut or castor oil. Thickening agents that can be used according to the nature of the base include soft paraffin, aluminum stearate, cetostearyl alcohol, propylene glycol, polyethylene glycols, polyphosphoesters, poly(lactic acids), hydroxyethyl celluloses, hydroxypropyl celluloses, cellulose gums, acrylate polymers, hydrophilic gelling agents, chitosan, polystyrene sulfonate, petrolatum, woolfat, hydrogenated lanolin, beeswax, and the like.

The ointments, pastes, creams, gels, and lotions can also contain excipients, such as animal and vegetable fats, oils, waxes, paraffins, starch, tragacanth, cellulose derivatives, polyethylene glycols, silicones, bentonites, silicic acid, talc, zinc oxide, and mixtures thereof. Powders and sprays can also contain excipients such as silicic acid, aluminum hydroxide, calcium silicates and polyamide powder, or mixtures of these substances. Solutions, suspensions or dispersions can be converted into aerosols or sprays by any of the known means routinely used for making aerosols for topical application. In general, such methods comprise pressurizing or providing a means of pressurizing a container of a solution, suspension or dispersion, usually with an inert carrier gas, and passing the pressured gas through a small orifice. Sprays and aerosols can also contain customary propellants, e.g., chlorofluorohydrocarbons or volatile unsubstituted hydrocarbons, such as butane and propane.

Excipients include compounds that promote skin absorption, such as dimethyl sulfoxide (DMSO), partial glycerides of fatty acids, and the like, present at levels up to about 10 wt % of the total formula weight. Examples of partial fatty acid glycerides include, but are not limited to IMWITOR 742 and IMWITOR 308 available from SASOL North America, Inc. of Houston, Tex. The topical formulations may also optionally include inactive ingredients to improve cosmetic acceptability, including but not limited to, humectants, surfactants, fragrances, coloring agents, emollients, fillers, and the like.

The topical compositions may also include other anti-infective agents, examples of which include bacitracin, neomycin, polymixin, beta-lactams, including penicillin, methicillin, moxalactam and cephalosporins, such as cefaclor, cefadroxil, cefamandole nafate, cefazolin, cefixime, cefinetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone, cefuroxime, cephalexin, cephalosporin C, cepahlosporin C sodium salt, cephalothin, cephalothin sodium salt, cephalothin dihydrate, cephapirin, cephradine, cefuroximeaxetil, loracarbef, and the like, glycopeptides, antibacterial enzymes, including anti-staphylococcal enzymes such as mutanolysin, lysozyme, or cellozyl muramidase, antibacterial antibodies, and other antibacterial peptides such as defensins. Essentially any anti-infective agent that is effective when applied topically can be used. Thus, the methods of the present invention for both treating active infections and decolonizing skin pathogen populations include methods in which lysostaphin or one or more lantibiotics are applied singularly or in combination, either with no other anti-infective agent, or with at least one other anti-infective agent.

The topical compositions can be administered directly by the dusting of a powder, spraying of an aerosol or by spreading a film of an ointment, cream, lotion, solution or gel to the desired area of the skin using the fingertips of the patient or a healthcare provider or other conventional application such as a swab or wipe. The product may be first applied to the skin and spread with the fingertips or an applicator or applied to the fingertips and spread over the skin. The compositions may also optionally first be coated on the surface of a topical applicator, such as a bandage, swab, moist woven or non-woven wipe and the like, which is then applied to the portion of the skin to receive the composition.

The topical compositions of the present invention can be prepared with base formulations that are essentially conventional to one of ordinary skill in the art with respect to the ingredients employed, quantities thereof, and methods of preparation, all of which require no further description. Topical compositions according to the present invention can also be prepared as a cream or lotion based on an emulsion formulation possessing heretofore unrecognized bactericidal activity, in addition to good skin compatibility and wound-healing properties that is particularly well-suited for formulation with anti-infective active ingredients.

Emulsions that are independently bactericidal according to the present invention on which anti-infective topical creams or lotions can be based are oil-in-water emulsions of an aqueous phase, an oil phase, and an emulsifier that is an inverse emulsion of a water-soluble polymer in an oil phase. Anti-infective topical formulations according to this embodiment of the present invention are prepared by adding an anti-infective active ingredient to the bactericidal emulsion. This embodiment of the present invention is not limited to the use of lysostaphin or lantibiotics such as nisin as the active ingredient. The independently bactericidal emulsions of the present invention will enhance the activity of essentially any anti-infective active ingredient that is effective when applied topically, while at the same time promoting wound healing and otherwise demonstrating good skin compatibility. It is not just the bactericidal activity of the emulsions that is unexpected. The inventive emulsions also unexpectedly enhance the activity of anti-infective agents beyond the contribution that would be expected from the activity of the emulsion and the activity of the anti-infective agent.

Anti-infective active ingredients for use with the independently bactericidal emulsions of the present invention for formulation as a topical cream or lotion include, in addition to lysostaphin and lantibiotics, other anti-infective active agents such as bacitracin, neomycin, polymixin, beta-lactams, including penicillin, methicillin, moxalactam and cephalosporins, such as cefaclor, cefadroxil, cefamandole nafate, cefazolin, cefixime, cefinetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone, cefuroxime, cephalexin, cephalosporin C, cepahlosporin C sodium salt, cephalothin, cephalothin sodium salt, cephalothin dihydrate, cephapirin, cephradine, cefuroximeaxetil, loracarbef, and the like, glycopeptides, anti-bacterial enzymes, including anti-staphylococcal enzymes such as mutanolysin, lysozyme or cellozyl muramidase, anti-bacterial antibodies, other anti-bacterial peptides such as defensins, and any other anti-infective active agent that is effective when applied topically. The present invention includes all possible multiple combinations of the above-listed anti-infective agents.

Using an inverse emulsion as an emulsifier permits formulation of topical creams and lotions under low temperature and shear conditions that do not degrade or denature heat- and -shear sensitive anti-infective agents such as lysostaphin and lantibiotics. When added to water, the emulsifier inverts to release the water-soluble polymer, thereby forming a polymeric gel base for the cream or lotion that serves to stabilize the oil-in-water emulsion on which the cream or lotion is based.

A suitable emulsifier is SEPIGEL 305 (Seppic, Inc., Fairfield, N.J.), which is an inverse emulsion of polyacrylamide in liquid paraffin. SIMUGEL 600 (Seppic, Inc.) may also be used, which is an inverse emulsion of polyacrylamide. Bactericidal topical cream formulations will contain about 2 and about 8% by weight, and more typically between about 3 and about 5% by weight of an inverse emulsion as the emulsifier. Bactericidal topical lotion formulations will contain between about 1 and about 10% by weight, and more typically between about 3 and about 5% by weight of an inverse emulsion as the emulsifier.

Bactericidal topical cream formulations according to the present invention will contain between about 2 and about 20% by weight, and typically between about 6 and about 10% by weight of an oil phase. Bactericidal topical lotion formulations will contain between about 1 and about 10% by weight, and typically between about 3 and about 5% by weight, of an oil phase. The oil phase can be a “hard fat,” which is a fatty acid triglyceride blend that is solid at room temperature, such as SOFTISAN 378, which is available from SASOL North America, Inc.

Bactericidal topical cream formulations will contain an aqueous phase at a level between about 70 and about 90% by weight, with an aqueous phase of about 80% by weight being typical. Bactericidal topical lotion formulations will contain an aqueous phase between about 85 and about 99% by weight, and typically between about 93 and about 98% by weight. In both the bactericidal topical cream and lotion formulations, the aqueous phase includes the anti-infective active ingredient and aqueous phase excipients, with the balance present as water.

Typical bactericidal topical cream and lotion formulations include in the aqueous phase as an excipient between about 2 and about 10% by weight of a skin absorption promoter such as DMSO or partial glycerides of fatty acids such as IMWITOR 308 and IMWITOR 742, both of which are available from SASOL North America, Inc. IMWITOR 308 is predominantly a glycerin monoester of caprylic acid. IMWITOR 742 is predominantly a blend of mono-, di- and triglcerides of capric and caprylic acids. Typical bactericidal cream and lotion formulations contain as aqueous phase excipients from about 0.25 to about 5% by weight, and more typically from about 1 and about 3% by weight of water soluble ethoxylated partial glycerides of fatty acids, such as SOFTIGEN 767, which is also available from SASOL North America, Inc.

According to one embodiment of the present invention a bactericidal cream or lotion formulation is prepared by pre-blending a hard fat such as SOFTISAN 78 with an emulsifier such as SEPIGEL 305 and an ethoxylated partial glyceride of fatty acids such as SOFTIGEN 767. The pre-blend can be heated slightly to liquify the hard fat at a temperature that will not degrade or denature the anti-infective agent to be added, typically between about room temperature and body temperature, and typically about 30° C. Higher temperatures, up to 100° C. and higher, can be used. However the mixture must then be permitted to cool before any ingredients susceptible to denaturing are added. Water, one or more anti-infective agents and an absorption promoter, if present, are separately pre-blended. The amount of water employed will determine whether the formulation is a cream or lotion. The two pre-blends are then combined with low-shear agitation to form an oil-in-water emulsion.

Emulsions in which the oil phase contains a hard fat and the aqueous phase contains ethoxylated partial glycerides of fatty acids have been found to possess, in addition to good skin compatibility and wound-healing properties, skin contact properties that promote the transport of the anti-infective active agent to the surface of a pathogen. Such formulations are storage stable at room temperature and maintain the topical product at the point of delivery for the length of time needed for treatment. Such emulsions also provide a protective matrix at the point of delivery that maintains the activity of the anti-infective agent.

The creams and lotions of the present invention that are independently bactericidal may be formulated with an amount of one or more anti-infective active agents between about 0.125 and about 10% by weight or more, recognizing that optimal dosages may differ only by 0.05% by weight within this range. Representative cream and lotion embodiments thus include every 0.05% by weight total concentration increment within this range of one or more anti-infective active agents.

Typical anti-infective active agents in formulations in which the cream or lotion is independently bactericidal include lysostaphin, lantibiotics, neomycin, bacitracin, combinations thereof, and the like. Typical combinations include lysostaphin and nisin.

As discussed above, the present invention is not limited to topical cream or lotion formulations, let alone formulations in which the topical cream or lotion is independently bactericidal. Topical formulations based on conventional sprays, mists, aerosols, lotions, creams, aqueous and non-aqueous solutions or liquids, oils, gels, ointments, pastes, unguents, emulsions and suspensions will contain an amount of lysostaphin, or a lantibiotic, or a combination of lysostaphin and a lantibiotic, and optionally one or more other anti-infective agents, in a total concentration of between about 0.125 and about 10% by weight or more, recognizing again that optimal dosages may differ only by 0.05% by weight, so that representative cream and lotion embodiments will include every 0.05% by weight concentration increment within this range. A typical formulation uses lysostaphin and nisin in combination.

Among the above-listed optional anti-infective active agents, those typically used include neomycin, bacitracin, and the like. A typical combination, which can also be used with cream and lotion formulations in which the base emulsion is independently bactericidal, is lysostaphin in combination with a subclinical concentration of bacitracin or neomycin. A subclinical concentration of bacitracin or neomycin is defined as that amount effective to prevent the emergence of lysostaphin-resistant S. aureus.

The topical compositions of the present invention are used to treat skin infections such as impetigo and wound infections such as surface wounds and penetrating wounds. Wounds suitable for treatment include wounds in skin abrasions, skin or surface cuts, decubiti, burns and surgical wounds. The topical compositions of the present invention can be used as well to decolonize populations of skin pathogens to prevent secondary, including the pre-treatment of areas prior to surgery or catheter insertion. For purposes of the present invention, skin pathogens are defined as including organisms that are native skin flora as well as non-native organisms, in both vegetative and non-vegetative states, including, but not limited to, S. aureus, S. epidermidis, P. aeruginosa, Strep. pyrogenes, and the like.

As used herein, “colonized” refers to the subclinical presence of skin pathogens, wherein “infected” refers to clinical infection of the skin or of a skin wound. Treatment methods according to the present invention thus include statistically significant reductions in the number of skin pathogen colonies present at clinical and subclinical levels.

Thus, one aspect of the invention is directed to a method of treating a patient with a clinical skin infection or wound infection, including patients with abrasions, skin or surface cuts, or decubiti, burn patients, patients with intravascular devices, and patients with impetigo, by administering the anti-infective topical compositions of the invention.

A skin infection or wound infection treatment method is effective if after application of an anti-infective topical composition to a human patient it causes a discernible or medically meaningful improvement in the progress of the infection, for example, as measured by the skin colonization assays described herein when applied to a sample taken from an active infection site subsequent to treatment. Accordingly, the “treatment” of an clinical skin or wound infection encompasses the administration of an effective amount of an anti-infective topical composition of the invention to the site of infection in one or more doses, with an effective amount being that amount sufficient to result in a medically meaningful, discernible, or statistically significant reduction, amelioration, alleviation, or eradication of the infection.

Another aspect of the invention is directed to a method of decolonizing sub-clinical skin pathogen populations to prevent secondary pathogen infections in patients susceptible thereto, such as patients with respiratory viral infections, transplant patients, HIV-infected patients, burn patients, patients with intravascular devices or foreign bodies, convalescent patients, and the like, by administering an effective amount of the anti-infective topical compositions of the invention to the skin in order to eliminate a primary source for subsequent infection.

An anti-infective topical composition according to the present invention is effective in decolonizing skin pathogen populations in a human patient if it causes a discernible decrease in the frequency of positive cultures or recoverable bacteria taken from a human patient who is already positive for one or more skin pathogens before the anti-infective topical composition of the invention is administered. An anti-infective topical composition of the invention “eradicates” skin pathogen colonies if after application there are no positive cultures taken from a human patient who had positive pathogen cultures prior to the application.

One embodiment of either treatment method is directed to the eradication, decolonization or blocking of skin colonization, and also the treatment of active skin and wound infections, in which any critical isolate of staphylococci is present, including any of the various capsule types, as well as strains that are resistant to methicillin, vancomycin, mupirocin and other antibiotics. The present invention thus has the added benefit of inhibiting the spread of antibiotic-resistant strains of staphylococci to the community by reducing the frequency and spread of staphylococcal infection.

Topical compositions are considered effective in decolonizing skin pathogen populations if the composition is able to either decrease the number of pathogen colonies on the skin of a mammal or the frequency of positive skin cultures for the presence of pathogens, when the composition is topically administered before, concurrently with, or after exposure to skin pathogens, whether that exposure results from the intentional installation of the pathogen or from general exposure. For instance, an anti-infective topical composition according to the present invention is considered effective to decolonize the skin if the number of bacterial colonies that can be grown from a sample of skin or a skin swab is decreased after topical administration of the topical compositions. An anti-infective topical composition according to the present invention effectively decolonizes the skin, as in the skin colonization assays described herein, when it decreases the number of colonies by at least 30% up to 100%. One hundred percent decolonization would be “eradication.”

An anti-infective topical composition according to the present invention is considered to “block” pathogen colonization if it is able to prevent the skin colonization of a mammal when the anti-infective topical composition is administered prior to or concurrently with, pathogen exposure, whether by intentional installation or otherwise. An anti-infective composition according to the present invention blocks colonization, as in the skin colonization assays described here, if no pathogen colonies can be grown from a sample of skin tissue, or skin swab, taken from a mammal treated with an anti-infective topical composition of the present invention for an extended period, such as 12 hours to 24 hours or longer after exposure.

Because a goal of the decolonization method of the invention, in which skin pathogen colonies are eradicated, decolonized or blocked, is to reduce the frequency of subsequent pathogen infections, the administration of an effective amount of the anti-infective topical composition of the invention includes that amount sufficient to demonstrate a medically meaningful, discernible, or statistically significant decrease in the likelihood of subsequent infection, for example, systemic infection, or infections at the site of trauma or surgery. Such demonstrations may encompass, for example, animal studies or clinical trials of patients at risk, including healthcare workers, newborns and premature infants, persons undergoing inpatient or outpatient surgery, burn victims, patients receiving in-dwelling catheters, stents, joint replacements, and the like, geriatric patients, patients with bed sores, and those with genetically, chemically or virally suppressed immune systems.

In a clinical setting, the presence or absence of skin pathogen colonies in a human patient is determined by culturing skin swabs on an appropriate bacterial medium often after an overnight enrichment step in a broth culture. The cultures are scored for the presence or absence of pathogen colonies. In this type of qualitative assay system, it may be difficult to distinguish between blocking of new colonies and decolonization of existing colonies. Once blocking or decolonization has occurred, the patient may be recolonized from an external source. For the purpose of qualitative assays, for example using skin swabs, an anti-infective topical composition according to the present invention “blocks” colonization if a human patient who at the time of treatment tests negative for skin pathogen colonies remains negative for an extended period, such as 12 to 24 hours or longer.

A “medically meaningful” treatment encompasses any treatment that improves the condition of a patient, improves the prognosis of a patient; improves the prognosis for a patient; reduces morbidity or mortality of a patient, reduces the likelihood of future colonization or infection, or reduces the incidence of morbidity or rates of mortality from the bacterial infections addressed herein, among a population of patients. The specific determination or identification of a “statistically significant” result will depend the exact statistical test used. One of ordinary skill in the art can readily recognize a statistically significant result in the context of any statistical test employed, as determined by the parameters of the test itself. Examples of these well-known statistical tests include, but are not limited to, the Chi-Squared Test, Student's test, F test, M test, Fisher Test Exact, Binomial Exact Test, Poisson Exact Test, one way or two way repeated measures analysis of variance, and calculation of correlation efficient (Pearson and Spearman).

In this context, a decolonization method is considered “medically meaningful” if the method produces (1) a statistically significant reduction in likelihood of future colonization or infection; (2) no colonization for at least 12 hours or more after final administration of the inventive anti-microbial topical composition; (3) a medically meaningful, discernible or statistically significant decrease in the number of skin pathogen colonies within at least 4 to about 24 hours or more after final administration of the inventive anti-infective topical composition; (4) a decrease in the frequency of positive cultures taken from the skin within at least 4 to about 24 hours or more after final administration; (5) continued activity of the anti-infective topical composition on the skin for at least 12 to about 48 hours or more after final administration; (6) eradication, decolonization or blockage of skin pathogens by a single dose up to ten doses or more of the inventive anti-infective topical composition; (7) any medically meaningful, discernible or statistically significant blocking or prophylaxis against future skin pathogen colonization; or (8) any medically meaningful, discernible, or statistically significant reduction in the likelihood of infection in the treated patient.

“Medically meaningful” treatment in the context of a clinical skin or wound infection includes any treatment that improves the condition of a patient; improves the prognosis for a patient; reduces morbidity or mortality of a patient; or reduces the likelihood of future colonization or infection of a patient; after administration of a single dose up to ten doses or more of the anti-infective topical composition of the invention.

In view of the disclosure provided, the administration of the anti-infective topical compositions of the invention is within the know-how and experience of one with skill in the art. In particular, the amount of the anti-infective topical composition required, combinations with appropriate carriers, the dosage schedule and amount may be varied within a wide range based on standard knowledge in the field without departing from the claimed invention. In one embodiment, the anti-infective topical composition may be administered once, twice, three times a day or more for at least one day or until the infection resolves, to a maximum of ten days. Such a dosing regimen would be effective on very young patients, very old patients, convalescing patients, pregnant mothers, those undergoing in-patient or out-patient invasive procedures (essentially any patient prior to release from a hospital), and patients suffering from various conditions that predispose them to primary or secondary skin infections.

A patient can be any human or any non-human mammal in need of prophylaxis or treatment of a clinical infection. Representative patients intended for topical administration are any mammal subject to S aureus or other skin pathogen infection or colonization, including humans and non-human animals, such as mice, rats, rabbits, dogs, cats, pigs, horses, primates, ruminants including milk and beef cattle, sheep, goats, buffalo and camels, as well as fur-bearing and herd animals, zoo and laboratory animals, farm, kenneled and stabled animals, domestic pets and veterinary animals.

The present invention is further illustrated by the following examples that teach those of ordinary skill in the art how to practice the invention. The following examples are merely illustrative of the invention and disclose various beneficial properties of certain embodiments of the invention. The following examples should not be construed as limiting the invention as claimed:

### EXAMPLES

Materials and Methods

Mouse Skin Infection Model:

Overnight cultures of S. aureus grown in TSB (SA5); ranging from 1 to 6×10⁹ CFUs/mL were centrifuged at 4000×g for 8 minutes and resuspended in an equal volume of phosphate buffered saline (PBS). SKH1(hrhr) hairless mice or shaved CF1 mice (Charles River) were sedated with 0.2 mL of ketamine (80 mg/kg) and xylazine (32 mg/kg) delivered intraperitoneally. The upper backs of the mice were scrubbed with a 70% alcohol wipe and allowed to dry. A single stitch of 4-0 silk sutures made by Ethicon and purchased from VWR Scientific, about 0.5 cm in length, was tied just below the neck on the upper back. Bacteria were swabbed over the stitch with a sterile, cotton-tipped applicator until the area was saturated with the solution. The next morning the mice were restrained and the stitches were removed from their backs.

Pathological Evaluation of Infection Model:

Hairless mice were infected as described with SA5. Mice were sacrificed on Day 1, 2, 3 or 10 by CO₂ asphyxiation. The 0.5 cm² section around the infection was excised and fixed in a PBS solution containing 10% formalin. Control mice were either completely untreated or had a suture stitched on their back but were not swabbed with bacteria. Both control groups were sacrificed on Day 1. Tissue sectioning and histopathology was performed at Taconic Anmed (Rockville, Md.). Samples were evaluated by a pathologist for an achantosis, subcutis inflammation or fibrosis and were scored for severity of change based on a scale from 0 (within normal limits) to 4 (severe).

Topical Cream:

The entire preparation was performed at room temperature and with simple hand mixing using a paddle-style weigh bar. 3 g of SEPIGEL 305, 8 g of SOFTISAN 378, and 2 g of SOFTIGEN 767 were added to a 250 mL glass beaker and mixed until a homogenous compound was formed, about one minute. Additional samples were prepared with 5 g of IMWITOR 308 and 5 g IMWITOR 742, respectively. 77 mL of sterile was then added all at once and mixed slowly until thickening began, about 1 to 2 minutes. The resulting cream was then mixed vigorously for 30 seconds to ensure uniformity and homogeneity. The final 10 mL of water, containing an appropriate amount of drug, was then added to the cream and mixed for 1 to 2 minutes.

In Vitro Efficacy of Topical Cream:

The anti-infective efficacy of lysostaphin (Biosynexus Incorporated, Gaithersburg, Md.) and nisin (Nutrition 21) formulated in the topical cream was tested for anti-infective activity against S. aureus and Pseudomonas aeruginosa (PA). Lysis of live SA5 and PA bacteria was measured by adding lysostaphin (0 to 10 μg/mL) or nisin (0 to 6 μg/mL) to an SA5 or PA suspension in PBS (% transmittance=40 at 650 nm; Spectonic 200+, Spectonic Instruments). Alternatively, 5 mM EDTA was added to the nisin-treated samples to determine if its presence enhanced the peptide's activity, particularly against gram-negative bacteria. The samples were incubated at 37° C. for 30 minutes and then streaked onto blood agar plates to determine viability after treatment. After overnight culture at 37° C., colonies were counted and compared to untreated samples.

Treatment of Infected Skin with Topical Cream:

Treatments to eradicate S. aureus infections in the mouse model were started on the morning after infection following removal of the suture (Day 1). Topical creams containing varying amounts of lysostaphin (0 to 0.5% w/w) and nisin (0 to 2% w/w) were swabbed over infected areas three times a day on Days 1 and 2 using a sterile, cotton-tipped applicator. The mice were sacrificed by CO₂ asphyxiation on the morning of Day 3 and a 0.5 cm² patch of skin around the infected area was excised. The skin sample was bisected and placed into a test tube containing 1 mL of PBS. Bacteria were dislodged from the skin sample by sonication: 2 minute treatments per sample with alternating 5 seconds at 2.5 W and 5 seconds at 0 W in a VIRSONIC 600 with microtip (Virtis). After mixing by vortex, 50 μL of each sample was streaked onto blood agar plates, incubated at 37° C. overnight, and the colonies were countered and compared to untreated controls.

Results

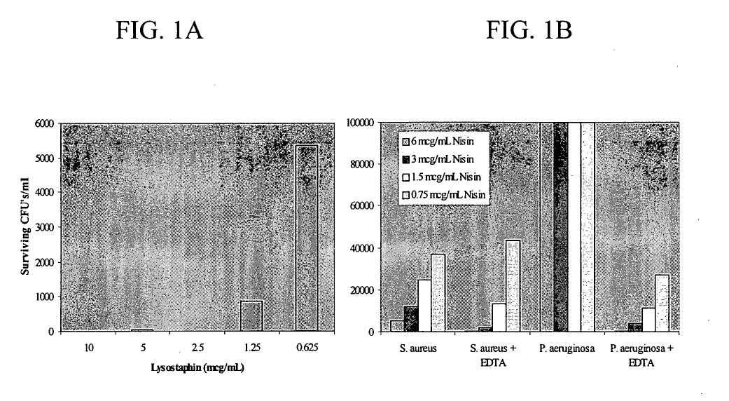

The in vitro efficacy of lysostaphin and nisin formulated into the topical cream was tested against S. aureus and P. aeruginosa. Nisin alone has been shown to have no activity against gram-negative bacteria. But if it is formulated with a chelator or surfactant, its bactericidal activity crosses over to many gram-negative strains. Therefore, nisin was formulated in the topical cream with or without 5 mM EDTA. Lysostaphin and nisin creams were both potent against S. aureus and the nisin cream containing EDTA had bactericidal activity against P. aeruginosa (FIGS. 1A and 1B). As expected, nisin alone had no activity against the gram-negative strain P. aeruginosa but addition of 5 mM EDTA to the formulation made the bacteria as equally sensitive to nisin as S. aureus. Addition of EDTA to the nisin cream formulation also enhanced the peptide's efficacy against S. aureus, especially at higher drug concentrations.

### Example 1

### In Vivo Efficacy of Lysostaphin Topical Cream

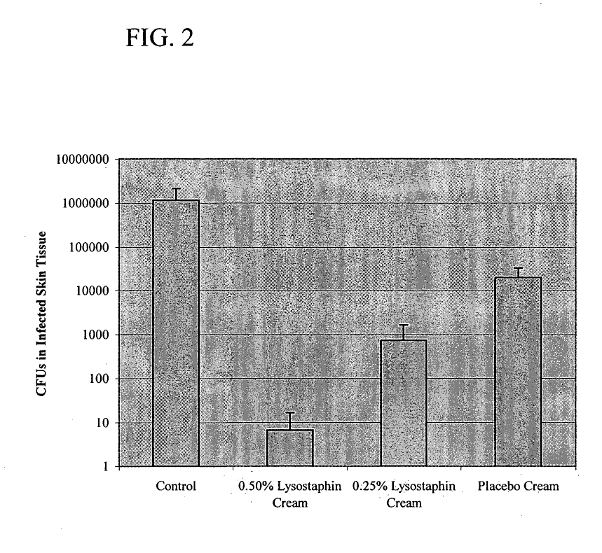

Lysostaphin was formulated into the topical cream to test its efficacy against S. aureus in the mouse skin infection model (FIG. 2). Six doses of a 0.5% w/w lysostaphin cream applied over two days eradicated infection in three of four mice and the remaining mouse had just a few colonies. The 0.25% w/w lysostaphin cream also demonstrated considerable activity, clearing one of three mice and reducing the infectious titer by over 3 logs in the other two, but was more than ten-fold less effective than the 0.5% w/w lysostaphin cream, indicating an exponential dose-response for this therapy. Surprisingly, the placebo cream also had considerable staphylococcal killing activity, reducing bacterial titers in the skin by about 100-fold. The activity attributable to lysostaphin creams is apparently a combinatorial or synergistic effect of killing by both lysostaphin and the base cream.

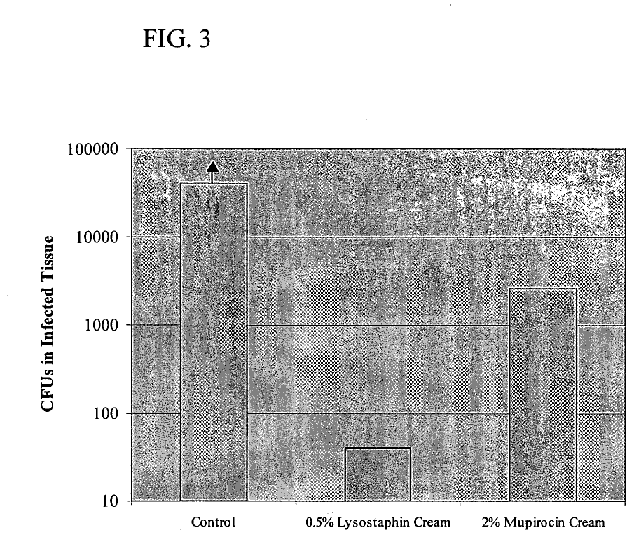

The efficacy of the 0.5% w/w lysostaphin cream was compared to a commercially available 2% mupirocin cream, BACTROBAN Cream (Glaxo SmithKline). On a mole basis there is 100-fold less lysostaphin in a 0.5% w/w cream than there is mupirocin in a 2% cream. However, the 0.5% w/w lysostaphin cream was far more effective in treating the mouse skin infection than 2% mupirocin cream after two days of 3 applications per day. (FIG. 3). The typical application for BACTROBAN Cream is 3 applications per day for 10 days, within clinical results expected within 3 to 5 days, so it is not entirely unexpected the mupirocin treated infections still show significant bacterial titers after just two day of therapy. In contrast, the lysostaphin cream was able to eradicate infection in one of three animals and reduce titers in the other two animals to fewer than 100 CFUs after two days of therapy, despite using 1/100th the amount of drug compared to mupirocin. These results demonstrate that lysostaphin is not only more potent than mupirocin in the skin infection model, but it also has a more rapid onset of action, factors that may decrease the probability that the lysostaphin-resistant strains of S. aureus will arise.

### Example 2

### In Vivo Efficacy of Nisin Topical Cream

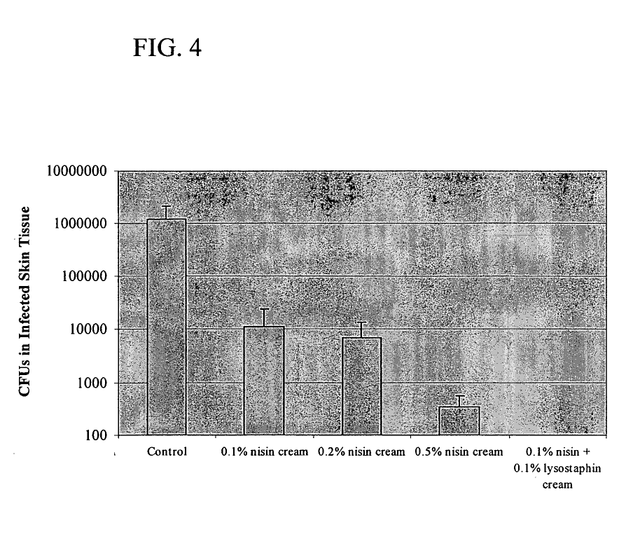

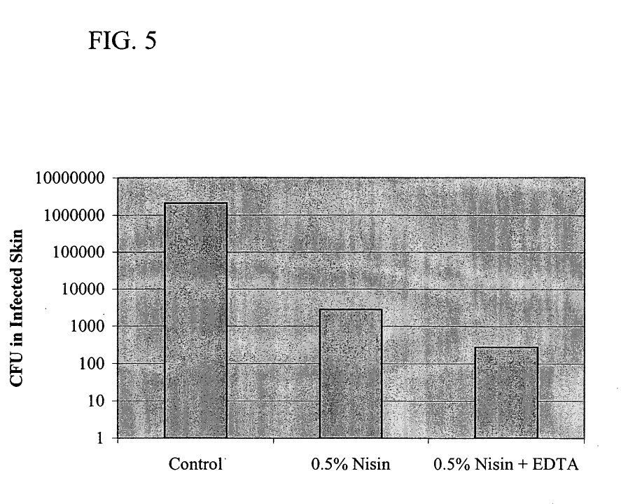

Nisin was formulated into the topical cream to test its efficacy against S. aureus and P. aeruginosa in the mouse skin infection model (FIGS. 4 and 5), which would help prevent the emergence of lysostaphin-resistant organisms. In combination with EDTA, nisin's bactericidal activity extends into gram-negative bacteria that are also significant causes of infectious skin disorders. Although the nisin cream is not as active as the lysostaphin cream (FIGS. 2 and 4), nisin still has significant activity against S. aureus after two days of therapy. The 0.5% w/w nisin cream did not eradicate infection in any of the animals but did reduce the bacterial titers by about 3.5 logs. More importantly, when small doses (0.1% w/w each) of lysostaphin and nisin were combined in a single cream formulation, the skin infection was completely eradicated in all animals tested. A 0.1% w/w cream of either lysostaphin or nisin alone was not sufficient to eradicate infection and only reduced the bacterial titers to about 10⁴ CFUs, compared to 10⁶ CFUs for the untreated controls.

Nisin was formulated with 5 mM EDTA in the topical cream to treat P. aeruginosa skin infections in the mouse model (FIG. 5). The infectious titers achieved with P. aeruginosa were not as high as those with S. aureus but the clinical observations of infection were more pronounced: stronger inflammatory response and more reddening over the infected area. Upon dissection of the infected skin, it was also apparent that the infection penetrates beyond the dermal layer of skin wherein the S. aureus infection was primarily contained within the subcutaneous skin layer. Nisin in combination with EDTA was very effective against P. aeruginosa skin infection, eradicating infection in 5 of 6 mice and significantly reducing the bacterial titer in the remaining mouse.

### Example 3

### In Vivo Efficacy of Topical Cream Without an Anti-infective Agent

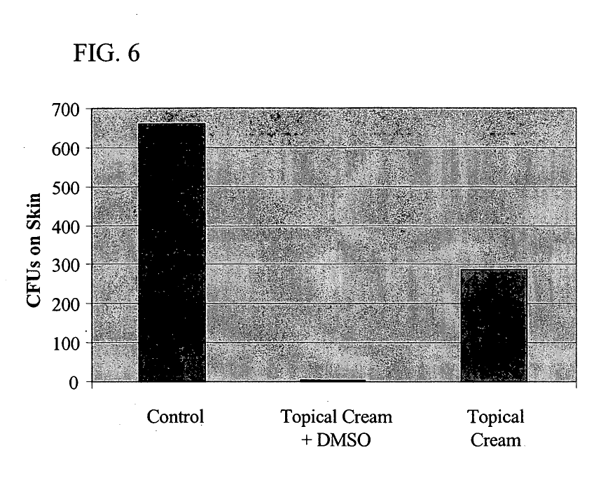

The in vivo efficacy of a cream formulation was demonstrated on a skin colonization model. The skin of hairless mice was colonized with S. aureus by swabbing bacteria over the surface of the skin (no wound or stitch). The colonized skin was treated 3 times a day for two days in either the topical cream base prepared in Example 1, or the topical cream base prepared in Example 1, plus 1% w/w DMSO. Lysostaphin in the topical cream only reduced skin colonization by about half whereas addition of the absorption enhancer DMSO to the cream without lysostaphin or any other anti-infective agent resulted in virtually the complete clearance of colonized bacteria (FIG. 6).

### Example 4

### In Vivo Efficacy of Topical Cream Without an Anti-Infective Agent

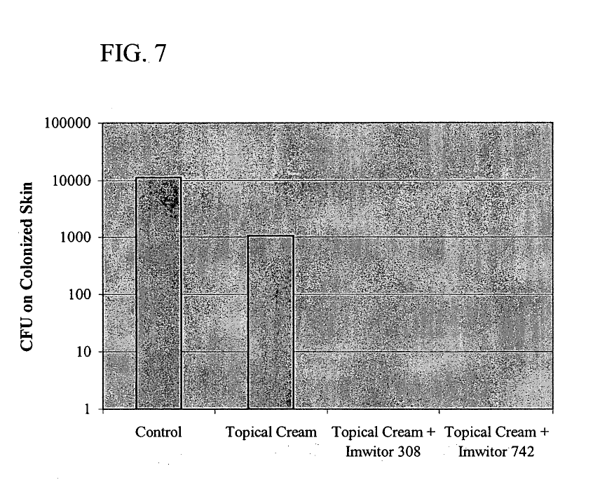

Example 3 was repeated, except that the DMSO topical cream was replaced with two topical cream samples, one in which the DMSO was replaced with IMWITOR 308, and one in which the DMSO was replaced with IMWITOR 742. Lysostaphin in the topical cream reduced skin colonization by about ten-fold, whereas the addition of the IMWITOR absorption enhancers to the cream resulted in virtually the complete clearance of colonized bacteria (FIG. 7).

### Example 5

### In Vivo Efficacy of Nisin Topical Cream for Skin Decolonization

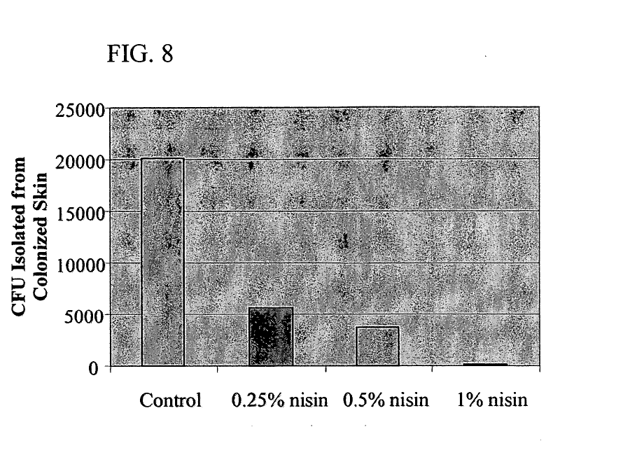

The nisin topical creams of Example 2 were formulated with 5% w/w IMWITOR 742. The skin of hairless mice was colonized with S. aureus as in Examples 3 and 4. The colonized skin was treated three times a day for 2 days. Nisin in this cream reduced skin colonization in a dose-dependent manner and achieved near complete clearance at a 1% w/w dose of nisin (FIG. 8).

### Example 6

### In Vitro Efficacy of Nisin Cream

A topical cream (100 g) was prepared by heating the waxes and oils to about 70° C. Nisin in an aqueous solution was added after the cream had cooled to room temperature. The following ingredients were mixed in a 250 mL glass beaker until a homogenous compound was formed:

| Ingredient | w/w |
|---|---|
| MIGLIOL¹ | 37.9% |
| SOFTISAN¹ | 25.5% |
| Petrolatum² | 28.9% |
| Paraffin, white, block³ | 3.6% |
| Beeswax⁴ | 3.6% |
| Aluminum stearate² | 0.5% |

¹Condea Vista
²Fluka
³Riedet de Haan
⁴Aldrich

To test the anti-infective activity of the nisin-based cream, and to compare this activity to that of the same concentration of nisin in PBS, 20 μL of a suspension of 9×10⁸ CFU/mL S. aureus was added to 1 g of cream and 1 mL of PBS at the nisin concentrations indicated in Table I. The samples were vortexed to mix and incubated at 37° C. for 60 minutes. 1 mL of mineral oil was added to the cream to facilitate pipetting and then serial dilutions were plated for enumeration of viable cells. The data in Table I show the viable cells for each nisin concentration.

TABLE I

| Nisin (μg/ml) | PBS (cfu/ml) | Cream (cfu/ml) |
|---|---|---|
| 0 | 70,000 | 50,000 |
| 0.5 | TNTC | 1082 |
| 2.5 | TNTC | 140 |
| 12.5 | 482 | 12 |

Example 1 demonstrates the dose-response curve of lysostaphin to be exponential in a cream formulation. The example also shows the cream based emulsion to be independently bactericidal. The example also demonstrates lysostaphin to be more potent than mupirocin in a skin infection model, and also to have a more rapid onset of action. Example 2 demonstrates the synergism between lysostaphin and nisin when the two compounds are used in combination. Example 2 also demonstrates the effectiveness of nisin in combination with EDTA against P. aeruginosa. Example 3 demonstrates the independent bactericidal activity of the topical cream base and the enhancement of this activity with DMSO. Example 4 demonstrates the enhancement of the bactericidal activity of the topical cream base by other absorption enhancers. Example 5 demonstrates the enhancement of nisin activity in a topical cream base by an absorption enhancer. Example 5 demonstrates the enhancement of nisin activity in a different cream base. Example 6 demonstrates the enhancement of nisin activity with surfactants in a gel base.

The foregoing examples thus demonstrate that topical forms of nisin and lysostaphin are highly effective in controlling populations of skin pathogens such as S. aureus and P. aeruginosa and that low concentrations together exhibit a synergistic level of activity. The examples also demonstrate the base emulsions of the invention to be independently bactericidal, thereby enhancing the activity of anti-infective active agents.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims:

What is claimed is:

1. A method of treating a skin or wound infection comprising topically applying to a patient in need thereof at the site of infection an effective amount of a topical composition comprising an effective amount of lysostaphin and/or one or more lantibiotics in a pharmaceutically acceptable carrier for topical application.

2. The method of claim 1, comprising treating a wound infection selected from infected abrasions, skin or surface cuts, burns or surgical incisions or decubiti.

3. The method of claim 1, wherein said topical composition comprises from about 0.10 to about 10.0 wt % lysostaphin selected from the group consisting of wild-type lysostaphin, lysostaphin mutants, variants and fragments, synthetic lysostaphins and recombinant lysostaphins, and has proteolytic activity against pentaglycine-containing bridges in the cell wall peptidoglycan of staphylococci.

4. The method of claim 1, wherein said topical composition comprises from about 0.10 to about 10.0 wt % of one or more lantibiotics selected from the group consisting of nisin, subtilin, epidermin, gallidermin, cinnamycin, duramycin, ancovenin, and Pep 5.

5. The method of claim 4, wherein said topical composition comprises nisin and a surfactant, and/or a chelating agent and/or carvacrol.

6. The method of claim 5, wherein said chelating agent comprises EDTA.

7. The method of claim 4, wherein said topical composition comprises a recombinant nisin variant.

8. The method of claim 4, wherein said topical composition comprises nisin and lysostaphin.

9. The method of claim 1, wherein said topical composition further comprises at least one anti-infective active agent other than lysostaphin or a lantibiotic.

10. The method of claim 9, wherein each anti-infective active agent or antibacterial enzyme is selected from the group consisting of beta-lactams, polymixin, glycopeptides, mutanolysin, lysozyme, cellozyl muramidase, antibacterial antibodies and antibacterial peptides.

11. The method of claim 9, wherein said topical composition further comprises at least one of bacitracin and neomycin.

12. The method of claim 1, wherein said pharmaceutically acceptable carrier for topical application is in the form of a spray, mist, aerosol, lotion, cream, aqueous or non-aqueous solution or liquid, oil, gel, ointment, paste, unguent, emulsion or suspension.

13. The method of claim 12, wherein said pharmaceutically acceptable carrier for topical application is an oil-in-water emulsion-based cream or lotion comprising an aqueous phase comprising ethoxylated partial glycerides of fatty acids, an oil phase comprising a hard fat, and an emulsifier that is an inverse emulsion of a water-soluble polymer in an oil phase.

14. The method of claim 13, wherein said aqueous phase comprises a skin absorption promoter selected from the group consisting of DMSO and partial fatty acid glycerides.

15. The method of claim 1, wherein said topical composition is a cream formulation comprising:
about 0.125 to about 10% by weight of lysostaphin and/or one or more lantibiotics;
about 2 to about 10% by weight of SOFTISAN 378;
about 0.25 to about 3% by weight of SOFTIGEN 767;
about 2 to about 8% by weight of SEIGEL 305 or SIMUGEL 600;
0 to about 10% by weight of IMWITOR 308 and/or IMWITOR 742; and
about 70 to about 90% by weight of water.

16. The method of claim 1, wherein said topical composition is coated on the surface of a topical applicator.

17. A topical composition comprising an effective amount of lysostaphin and/or or one or more lantibiotics in a pharmaceutically acceptable carrier for topical application.

18. The topical composition of claim 17, wherein the amount of lysostaphin or lantibiotic is effective to treat skin infections or infected wounds selected from infected abrasions, skin or surface cuts, burns or surgical incisions or decubiti.

19. The topical composition of claim 17, comprising from about 0.10 to about 10.0 wt % of lysostaphin selected from the group consisting of wild-type lysostaphin, lysostaphin mutants, variants and fragments, synthetic lysostaphins and recombinant lysostaphins, and has the proteolytic activity against pentaglycine-containing bridges in the cell wall peptidoglycan of staphylococci.

20. The topical composition of claim 17, comprising from 0.10 to about 10.0 wt % of one or more lantibiotics selected from the group consisting of nisin, subtilin, epidermin, gallidermin, cinnamycin, duramycin, ancovenin, and Pep 5.

21. A topical composition according to claim 20, comprising nisin, and a surfactant, or a chelating agent or carvacrol.

22. The topical composition of claim 21, wherein said chelating agent is EDTA.

23. The topical composition of claim 20, comprising a recombinant nisin variant.

24. A topical composition according to claim 20, comprising lysostaphin and nisin.

25. The topical composition of claim 17, further comprising at least one anti-infective active agent other than lysostaphin or a lantibiotic.

26. The topical composition of claim 25, wherein each anti-infective active agent is selected from the group consisting of beta-lactams, polymixin, glycopeptides, mutanolysin, lysozyme, cellozyl muramidase, antibacterial antibodies and antibacterial peptides.

27. The topical composition of claim 25, wherein said anti-infective active agent comprises at least one of bacitracin and neomycin.

28. The topical composition of claim 17, wherein said pharmaceutically acceptable carrier for topical application is in the form of a spray, mist, aerosol, lotion, cream, aqueous or non-aqueous solution or liquid, oil, gel, ointment, paste, unguent, emulsion or suspension.

29. The topical composition of claim 28, wherein said pharmaceutically acceptable carrier for topical application is an oil-in-water emulsion-based cream or lotion comprising an aqueous phase comprising ethoxylated partial glycerides of fatty acids, an oil phase comprising a hard fat, and an emulsifier that is an inverse emulsion of a water-soluble polymer in an oil phase.

30. The topical composition of claim 29, wherein said aqueous phase comprises a skin absorption promoter selected from the group consisting of DMSO and partial fatty acid glycerides.

31. A topical composition according to claim 17, in the form of a topical cream comprising:
about 0.125 to about 10% by weight of lysostaphin and/or one or more lantibiotics;
about 2 to about 10% by weight of SOFTISAN 378;
about 0.25 to about 3% by weight of SOFTIGEN 767;
about 2 to about 8% by weight of SEIGEL 305 or SIMUGEL 600;
0 to about 10% by weight of IMWITOR 308 and/or IMWITOR 742; and
about 70 to about 90% by weight of water.

32. The topical composition of claim 17, coated on the surface of a topical applicator.

33. A method of decolonizing skin pathogen populations comprising topically applying to a patient in need thereof at a site requiring decolonization an effective amount of the topical composition of claim 17.

34. The method of claim 33, wherein said topical composition comprises from about 0.10 to about 10.0 wt % of lysostaphin selected from the group consisting of wild-type lysostaphin, lysostaphin mutants, variants and fragments, synthetic lysostaphins and recombinant lysostaphins, and has proteolytic activity against pentaglycine-containing bridges in the cell wall peptidoglycan of staphylococci.

35. The method of claim 33, wherein said topical composition comprises from about 0.10 to about 10.0 wt % of one or more lantibiotics selected from the group consisting of nisin, subtilin, epidermin, gallidermin, cinnamycin, duramycin, ancovenin, and Pep 5.

36. The method of claim 35, wherein said topical composition comprises nisin and a surfactant, or a chelating agent or carvacrol.

37. The method of claim 36, wherein said chelating agent comprises EDTA.

38. The method of claim 35, wherein said topical composition comprises a recombinant nisin variant.

39. The method of claim 35, wherein said topical composition further comprises lysostaphin.

40. The method of claim 33, wherein said topical composition further comprises at least one anti-infective active agent other than lysostaphin or a lantibiotic selected from the group consisting of beta-lactams, polymixin, glycopeptides, mutanolysin, lysozyme, cellozyl muramidase, antibacterial antibodies and antibacterial peptides.

41. The method of claim 33, wherein said topical composition further comprises at least one of bacitracin and neomycin.

42. The method of claim 33, wherein said pharmaceutically acceptable carrier for topical application is in the form of a spray, mist, aerosol, lotion, cream, aqueous or non-aqueous solution or liquid, oil, gel, ointment, paste, unguent, emulsion or suspension.

43. The method of claim 42, wherein said pharmaceutically acceptable carrier for topical application is an oil-in-water emulsion-based cream or lotion comprising an aqueous phase comprising ethoxylated partial glycerides of fatty acids, an oil phase comprising a hard fat, and an emulsifier that is an inverse emulsion of a water-soluble polymer in an oil phase.

44. The method of claim 43, wherein said aqueous phase comprises a skin absorption promoter selected from the group consisting of DMSO and partial fatty acid glycerides.

45. The method of claim 33, wherein said topical composition is a cream formulation comprising:
about 0.125 to about 10% by weight of lysostaphin and/or one or more lantibiotics;
about 2 to about 10% by weight of SOFTISAN 378;
about 0.25 to about 3% by weight of SOFTIGEN 767;
about 2 to about 8% by weight of SEIGEL 305 or SIMUGEL 600;
0 to about 10% by weight of IMWITOR 308 and/or IMWITOR 742; and
about 70 to about 90% by weight of water.

46. The method of claim 33, wherein said topical composition is coated on the surface of a topical applicator.

47. An oil-in-water emulsion-based topical cream or lotion composition comprising an aqueous phase comprising ethoxylated partial glycerides of fatty acids, an oil phase comprising a hard fat, an emulsifier that is an inverse emulsion of a water-soluble polymer in an oil phase, and an effective amount of one or more anti-infective active agents in said aqueous phase or said oil phase.

48. The topical composition of claim 47, wherein said aqueous phase comprises a skin absorption promoter selected from the group consisting of DMSO and partial fatty acid glycerides.

49. The topical composition of claim 47, coated on the surface of a topical applicator.

50. The topical composition of claim 47, wherein said emulsifier is an inverse emulsion of polyacrylamide in liquid paraffin.

51. The topical composition of claim 47, wherein at least one anti-infective active agent is selected from the group consisting of beta-lactams, polymixin, glycopeptides, mutanolysin, lysozyme, cellozyl muramidase, antibacterial antibodies and antibacterial peptides.

52. The topical composition of claim 47, wherein each anti-infective active agent is selected from the group consisting of lysostaphin, lantibiotics, bacitracin and neomycin.

53. The topical composition of claim 51, wherein said anti-infective active agents comprise lysostaphin and nisin.

54. The topical composition of claim 47, in the form of a topical cream comprising: wherein said topical composition is a cream formulation comprising:
about 0.125 to about 10% by weight of lysostaphin and/or one or more lantibiotics;
about 2 to about 10% by weight of SOFTISAN 378;
about 0.25 to about 3% by weight of SOFTIGEN 767;
about 2 to about 8% by weight of SEIGEL 305 or SIMUGEL 600;
0 to about 10% by weight of IMWITOR 308 and/or IMWITOR 742; and
about 70 to about 90% by weight of water.
